(19) United States
(12) Patent Application Publication
(10) Pub. No.: US 2019/0337521 A1
(43) Pub. Date: Nov. 7, 2019

(54) METHOD AND CONTROL UNIT FOR OPERATING A SELF-DRIVING CAR

(71) Applicant: ZF FRIEDRICHSHAFEN AG, Friedrichshafen (DE)

(72) Inventor: Tobias Stauber, Friedrichshafen (DE)

(73) Assignee: ZF FRIEDRICHSHAFEN AG, Friedrichshafen (DE)

(21) Appl. No.: 16/404,201

(22) Filed: May 6, 2019

(30) Foreign Application Priority Data
May 7, 2018 (DE) 10 2018 207 069.5

(51) Int. Cl. B60W 40/08; A61B 5/0205; A61B 5/1455; A61B 5/00

(52) U.S. Cl. CPC B60W 40/08; A61B 5/0205; G05D 1/0088; A61B 5/4803; A61B 5/14552

(57) ABSTRACT

The invention relates to a control unit for autonomous driving (18) comprising a processor (40) configured to rate an executed or upcoming driving maneuver (M1-M4) on the basis of physiological measurements

### FIELD

The present invention relates to a method and a control unit for operating an autonomous vehicle.

### DESCRIPTION OF RELATED ART

An autonomous vehicle is a vehicle that can drive, steer and park without a human driver. With autonomous driving, the control system of the vehicle fully or substantially assumes the role of the driver. Autonomous vehicles can detect their environment with various sensors, determine their position and the positions of other road users from the acquired data, and drive to a destination and react appropriately in street traffic by means of the control system and navigation software of the vehicle.

As can be derived from DE 10 2014 212 746, the use of automation has increased in driving street vehicles such as automobiles and trucks through the advances in sensor technology (e.g. object detection and tracking), control algorithms and data infrastructures.

In addition to the contribution to mobility, particularly for handicapped persons and the elderly, autonomous driving reduces the risk of accidents resulting from slow reaction times, drowsiness, distractions and other human factors.

On the other hand, autonomous (self-driving) vehicles may exhibit a driving behavior that is significantly different than that of vehicles driven by a person, e.g. regarding braking behavior and maneuvering in street traffic. As a result, some users and passengers in autonomous vehicles may find the reactions of the vehicle to be unexpected, unpleasant, or even frightening.

Based on this, the international patent application WO 2016/070981 A1 discloses a system and a method for monitoring the state of health of a vehicle occupant. The system comprises a control unit comprising a receiver for wireless reception of physiological parameters of at least one portable unit, which comprises one or more sensors for determining one or more physiological parameters of the vehicle occupant, and a diagnosis module configured to derive information regarding the state of health, the well being, or abnormal occurrences on the basis of the received physiological parameters. The control unit is also configured to inform the vehicle occupants of the state of health via at least one output unit, and to initiate at least one of the following steps: adapt vehicle functions to the state, or propose or interactively execute measures that serve to improve the state, via at least one output unit.

### SUMMARY

Based on this, the fundamental object of the invention is to create a method and a control unit for operating an autonomous vehicle that optimize the driving behavior of the vehicle.

This object is achieved by the control unit for autonomous driving according to claim 1 and the method according to claim 10. Further advantageous embodiments of the invention can be derived from the dependent claims and the following description of preferred exemplary embodiments of the present invention.

According to the exemplary embodiments described below, a control unit is created for autonomous driving, which comprises a processor that is configured to evaluate an executed or upcoming driving maneuver based on physiological measurements.

A control unit for autonomous driving can be a control device (Eng.: ECU=electronic control unit, or ECM=electronic control module). The control unit for autonomous driving (e.g. an “autopilot”) can be used, for example, in an autonomous vehicle, such that it can drive, steer and park, entirely or partially without the input of a human driver. The control unit can be located in the vehicle, or it can be outside or partially outside the vehicle. It can be an algorithm, for example, that runs on a server or a cloud system. By way of example, measurements can be obtained in a vehicle, in which the well being of the vehicle occupants is determined based on the obtained measurements, and the results are then returned to the vehicle. In addition to the measurements, the vehicle can also transmit possible driving maneuvers to the server or the cloud system, and an evaluation of the driving maneuver can take place in the server or the cloud based on the measurements or the states of vehicle occupants. Furthermore, the selection of the optimal driving maneuver can take place outside the vehicle, on the server or in the cloud, such that the autonomous vehicle is merely transmitted the driving maneuver selected on the basis of the measurements. Accordingly, it is provided that the control unit, or control logic can also be located entirely or partially outside a vehicle.

The processor can be a computing unit, such as a central processing unit (CPU=central processing unit), for example, that executes program instructions.

A driving maneuver can relate to a specific driving situation, for example, that represents an objective given spatial and temporal constellation of the traffic related contributing factors of the functional environment of a vehicle. Driving maneuvers can be predefined in the control unit, and set, for example, through contextual information (e.g. the position of the vehicle, navigation context, etc.) and vehicle operating parameters (speed, transverse acceleration, torque). Driving maneuvers can be classified according to certain categories. Examples of driving maneuvers are “exiting a highway,” “cornering,” “approaching a traffic light,” “tailgating,” “avoiding the shoulder,” “braking in a curve,” “passing on the highway,” “passing on single-lane roads,” accelerating on ramps,” “decelerating on ramps,” “approaching,” “driving over uneven ground or speed bumps,” etc. Driving maneuvers of one category can be subdivided into numerous driving maneuver variations, e.g. “cornering with a transverse acceleration of 2 m/s2” as a first variation, “cornering with a transverse acceleration of 3 m/2” as a second variation, etc. Instead of transverse acceleration, the driving maneuver can also be subdivided into driving maneuver variations regarding the size of the corning radius and the driving speed. Such driving maneuvers are perceived differently by everyone, and regarded as pleasant or unpleasant.

The control unit can be configured to control the vehicle at least partially on the basis of a person-specific evaluation of a driving maneuver, and thus to optimize the driving behavior of the vehicle. By way of example, the processor in the control unit can be configured to evaluate a completed or upcoming driving maneuver for autonomous driving, in that a driving maneuver is assigned a rating based on physiological measurements regarding a vehicle occupant. The rating can be defined, for example, by a parameter, a numerical value, etc. By way of example, various ratings can be predefined in the control unit for autonomous driving, wherein each rating is allocated to a well being of a vehicle occupant.

The physiological measurements can relate, for example, to an occupant of an autonomous vehicle, e.g. a vehicle operator or other occupants, such as a passenger in the front or rear seats of the vehicle. By way of example, a driving maneuver can be an autonomous vehicle braking at a red light. The driving maneuver can be carried out in that the vehicle brakes well before reaching the red light, “rolls” (coasts), recuperates or “coasts” (clutch is disengaged), and the vehicle decelerates. If the light changes to green, the vehicle must be accelerated in order to cross the intersection while it is still green. The occupants of the vehicle may find the early deceleration and subsequent acceleration unpleasant, and react accordingly. The present control unit can detect such feelings, and rate the driving maneuver accordingly. This has the advantage that unpleasant responses by the vehicle occupants can be prevented by the control unit for autonomous driving.

The physiological measurements preferably relate to health data/biological data, e.g. pulse rate, skin conductance, oxygen saturation in blood, blood pressure, concentration of a stress hormone, e.g. adrenaline concentration, etc. Different priorities can be assigned to the physiological measurements. By way of example, the measurement of the pulse rate can be given a higher priority than the measurement of the skin conductance. Alternatively or additionally, the physiological measurements can relate to data obtained through image analysis or speech analysis. Thus, an image recognition can be carried out on the basis of images recorded by an interior camera in the vehicle. The image recognition can be based on gesture recognition, for example. By way of example, an angry gesture by a vehicle occupant, or when the occupant's eyes have been closed for a longer period of time, can be detected through gesture recognition, and conclusions regarding a state of fear or stress can be drawn. Interior cameras can thus analyze gestures and determine the well being of the vehicle occupants. Alternatively, states of health or illness can be determined on the basis of skin color, body shape (pregnancy), blood, etc. By way of example, vital signs, in particular the pulse rate, respiratory rate and breathing volume, can be determined from differences in the hue of the light reflected by the skin over time. This process is also referred to as photoplethysmography. The fundamentals of the process are described, by way of example, in the article by Ming-Zher poh, Daniel J. McDuff, and Rosalind W. Picard, “Non-contact, automated cardiac pulse measurements using video imaging and blind source separation,” Opt. Express 18, 10762-10774 (2010).

Conversation in the automobile can also be analyzed in order to obtain information regarding the manner of driving from which conclusions can be drawn regarding the well being of the vehicle occupants.

The physiological measurements can be recorded by one or more user devices, e.g. a smartwatch, and sent to the control unit for autonomous driving. The heart rate is evaluated by smartwatches and fitness bracelets, and further data are acquired and derived. User devices such as a smartwatch, a cellular telephone and other devices can acquire data and enable statements regarding the well being and/or health of the vehicle occupants. Alternatively, the physiological measurements can be recorded by built-in sensors, e.g. capacitive sensors integrated in the driver seat, the headrests, armrests, safety belts, etc. that can detect the heart rate, respiration, or brain waves, or by electrodes on the steering wheel for detecting the heart rate. The use of neural-headsets, which record brain activity and emotions, or breast-pulse belts, implanted chips, etc. is likewise conceivable. The physiological measurements or health data are received from the user device in this manner by the control unit for autonomous driving. Alternatively, the user device can also analyze or process the physiological measurements or health data itself. In another alternative, the health data can also be analyzed and processed by a cloud, and then returned to the vehicle (in real-time or in post-processing).

The physiological measurements are preferably determined at a time that correlates to the execution of the driving maneuver. By way of example, the physiological measurements are determined during the execution of the driving maneuver or shortly after executing the driving maneuver. The physiological measurements can be determined continuously, and associated with corresponding driving maneuvers, for example.

The processor can also be configured to create a user profile based on evaluated driving maneuvers. Person-specific ratings of predefined driving maneuvers or driving maneuver variations can be stored in the user profile, for example.

The processor can also be configured to rate an upcoming driving maneuver based on the user profile that has been created. By way of example, the processor can be configured to rate numerous variations of driving maneuvers based on stored ratings in a user profile, and select an optimal variation based on the rating.

The user profile can also comprise further information. By way of example, in current vehicles it is possible to adjust the chassis components, thus to set the chassis to tight/sporty, comfortable, etc. A sporty user profile has chassis components set for sports driving, such that uneven ground can be felt in the vehicle. An economical user profile has the chassis components set for comfort, such that the same uneven ground is felt to a lesser extent. Such settings in the user profile can be used in evaluating driving maneuvers, e.g. in that the ratings assigned to the driving maneuver variations are assessed accordingly. Thus, when a sporty profile is set, cornering with high transverse acceleration can have a higher rating than with an economic setting of the user profile, if this behavior is desired by the manufacturer or the occupants.

User profiles can be based on specific days and/or times of day, or a destination (e.g. comfortable in the morning, fast in the afternoon, comfortable on weekends, etc.)

The control unit for autonomous driving can activate a control unit for a steering system, a control unit for a braking system and/or a control unit for a drive train in accordance with the selected driving maneuver variation, such that the selected driving maneuver or the selected driving maneuver variation is carried out.

The processor can also be configured to detect relative changes in the physiological measurements and to rate a driving maneuver based on the relevant changes in the physiological measurements.

The processor can also be configured to check whether a change in the physiological measurements can be attributed to a driving maneuver or some other reason, and then only rate the driving maneuver when other reasons can be dismissed. By way of example, another reason for the physiological measurement can be identified by accessing a calender application. If, for example, the control unit identifies an upcoming meeting in the calender, and the location of the meeting corresponds to the destination of the vehicle or the workplace of the occupant, the control unit can take this information into account when rating driving maneuvers.

Thus, it can be assumed, for example, that the pulse of the driver will increase as the appointment approaches, such that the pulse increase can no longer be attributed to just the driving maneuver, and for this reason, the driving maneuver is not evaluated, or the sense of urgency attributed to the appointment is incorporated in the evaluation.

Alternatively, the control unit can also be configured to switch to a sporty manner of driving when it identifies the urgency of the appointment, such that the occupant is given the impression that he will arrive at his destination more quickly.

The invention also relates to a method for autonomous driving in which a completed or upcoming driving maneuver is rated on the basis of physiological measurements. The method can be a computer-implemented method.

### BRIEF DESCRIPTION OF THE DRAWINGS

Embodiments shall now be described by way of example and with reference to the attached drawings, wherein:

FIG. 1 shows an overall scenario of an exemplary embodiment of a vehicle control system;

FIG. 2 shows a block diagram schematically illustrating the configuration of an autonomous vehicle according to an exemplary embodiment of the present invention;

FIG. 3 shows a block diagram illustrating an exemplary configuration of a portable user device;

FIG. 4 shows a block diagram illustrating an exemplary configuration of a control device for autonomous driving;

FIG. 5 shows predefined rating values assigned to specific states of being of the vehicle occupants according to an exemplary embodiment of the invention;

FIG. 6a shows how various ranges of a measured heart rate are assigned to predefined rating values according to an exemplary embodiment of the invention;

FIG. 6b shows how various ranges of a measured heart rate combined with a measured skin conductance are assigned to predefined rating values according to an alternative exemplary embodiment of the invention;

FIG. 6c shows how various ranges of a heart rate are assigned in a user-specific manner to rating values according to another alternative exemplary embodiment of the invention;

FIG. 6d shows how various ranges of a heart rate are assigned to driving mode-specific rating values according to yet another alternative exemplary embodiment of the invention;

FIG. 7a shows a schematic illustration of numerous predefined driving maneuver variations for a driving maneuver “cornering;”

FIG. 7b shows a schematic illustration of numerous predefined driving maneuver variations for a driving maneuver “highway driving;”

FIG. 8 shows an exemplary table comprising a temporal sequence of predefined and rated driving maneuvers;

FIG. 9 shows an exemplary graph of the rating values determined by the control unit for autonomous driving for the temporal sequence of the driving maneuver in FIG. 8;

FIG. 10 shows a flow chart that illustrates a method for creating a user profile by the control unit for autonomous driving, in which the physiological measurements are rated in an absolute manner;

FIG. 11 shows a flow chart that illustrates a method for creating a user profile by the control unit for autonomous driving, in which the physiological measurements are rated in a relative manner (based on changes);

FIG. 12 shows an exemplary table that illustrates a user profile for the driving maneuver variations of a driving maneuver “cornering,”

FIG. 13 shows a flow chart that illustrates a rating of possible driving maneuver variations of an upcoming driving maneuver based on a user profile.

### DETAILED DESCRIPTION

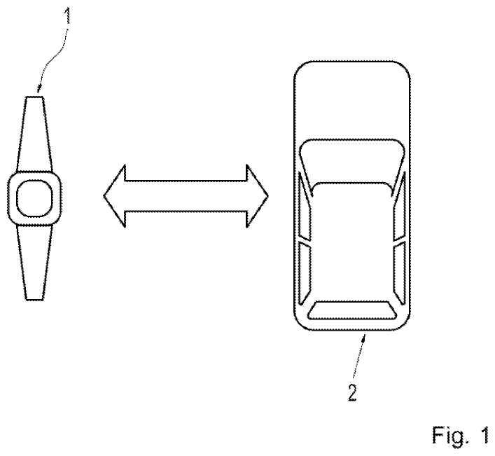

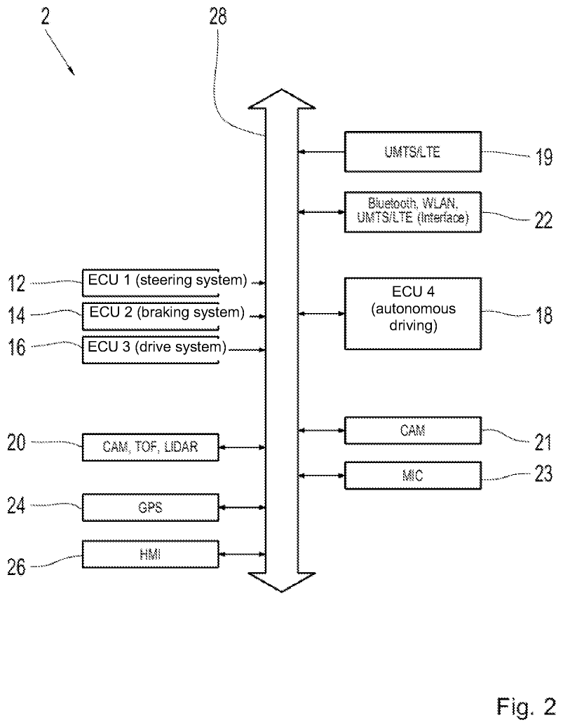

FIG. 1 shows an overall scenario of an exemplary embodiment of a vehicle control system. A portable user device 1, in this case a smartwatch, is used as a sensor device that provides physiological measurements of a vehicle occupant. The portable user device 1 is worn by a vehicle occupant, e.g. on the wrist or some other arbitrary suitable place on the body. The portable user device 1 communicates with an autonomous (or semi-autonomous) vehicle 2. The portable user device 1 is configured to take one or more physiological measurements regarding the vehicle occupant and to transmit them via a wireless interface or a hard-wired interface to a control unit 18 for autonomous driving (see FIG. 2) in an autonomous vehicle 2, such that the control unit 18 for autonomous driving can use the recorded data to rate a driving maneuver carried out by the vehicle 2 at the time the physiological measurements are recorded based on the recorded physiological data of the vehicle occupant.

FIG. 2 shows a block diagram schematically illustrating the configuration of a vehicle 2 that has a control unit for autonomous driving according to an exemplary embodiment of the present invention. The autonomous vehicle 2 comprises numerous electronic components, which are interconnected via a vehicle communications network 28. The vehicle communications network 28 can be a standard vehicle communications network built into the vehicle, e.g. a CAN bus (controller area network) a LIN bus (local interconnect network), a LAN bus (local area network), a MOST bus, a FlexRay bus (registered trademark), and/or an Ethernet bus system, etc.

In the example shown in FIG. 2, the autonomous vehicle 2 comprises a control device 12 (ECU 1). This control device 12 controls a steering system. The steering system relates to the components that enable a directional control of the vehicle.

The autonomous vehicle 2 also comprises a control unit 14 (ECU 2) that controls a braking system. The braking system relates to the components that enable a braking of the vehicle.

The autonomous vehicle 2 furthermore comprises a control unit (ECU 3) that controls a drive train. The drive train relates to the drive components of the vehicle. The drive train can comprise a motor, a transmission, a drive/propulsion shaft, a differential and an axle drive.

The autonomous vehicle 2 also comprises a control unit for autonomous driving 18 (ECU 4). The control unit for autonomous driving 18 is configured to drive, steer and park the autonomous vehicle 2 such that it is operated entirely or partially without the influence of a human driver.

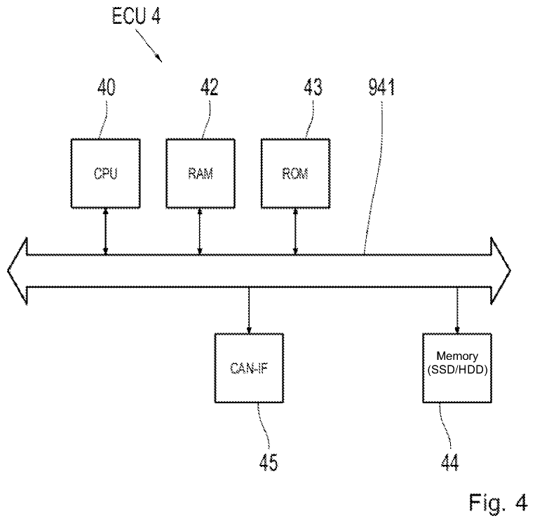

The control unit for autonomous driving 18, which is illustrated in FIG. 4 and described in greater detail in reference thereto, controls one or more vehicle subsystems while the vehicle is operated in the autonomous mode, specifically the braking system 14, the steering system 12 and the drive system 16. For this, the control unit for autonomous driving 18 can communicate via the vehicle communication network 28 with the corresponding control units 12, 14, 16. The control units 12, 14, and 16 can also receive vehicle operating parameters from the aforementioned vehicle subsystems that they record by means of one or more vehicle sensors. The vehicle sensors are preferably sensors that record a state of the vehicle or a state of vehicle components, in particular the state of movement. The sensors can comprise a vehicle speed sensor, a yaw rate sensor, an acceleration sensor, a steering wheel angle sensor, a vehicle load sensor, temperature sensors, pressure sensors, etc. By way of example, sensors can also be located along the brake lines for outputting signals that indicate the brake fluid pressure at various points along the hydraulic brake lines. There can be other sensors in the proximity of the wheels, which record the wheel speed and the brake pressure applied to the wheel.

The vehicle sensor system of the autonomous vehicle 2 also comprises a satellite navigation unit 24 (GPS unit). It should be noted that in the context of the present invention, GPS stands for any global navigation satellite system (GNSS), such as GPS, A-GPS, Galileo, GLONASS (Russia), Compass (China), IRNSS (India), etc.

When an operating state is activated for autonomous driving by the control system or the driver, the control unit 18 for autonomous driving determines parameters for the autonomous operation of the vehicle (e.g. target speed, target torque, distance to the vehicle in front, steering procedure, etc.) on the basis of available data regarding a predefined route and vehicle operating data recorded by means of vehicle sensors that are sent to the control unit 18 from the control units 12, 14, 16.

The autonomous vehicle 2 also comprises one or more environment sensors 20 that are configured to record the environment of the vehicle, wherein the environment sensors 20 are installed on the vehicle and record objects or states in the environment of the vehicle self-sufficiently, i.e. without outside information signals. These include cameras, radar sensors, lidar sensors, ultrasound sensors, etc. The environment sensors 20 can be located inside or outside the vehicle (e.g. on the outer surface of the vehicle).

The autonomous vehicle 2 also comprises one or more interior cameras 21, which provide image data on the occupants of the vehicle 2. Based on the image data on the vehicle occupants, the control unit for autonomous driving 18 requests further information regarding the well being of the vehicle occupants, and incorporates this knowledge in the evaluation of driving maneuvers, in order to adapt the manner of driving to the well being, or a profile of the occupant. If it is determined, for example, that a vehicle occupant has made an angry gesture, or closed his eyes over a longer period of time, conclusions can be drawn regarding the state of fear or stress. The skin temperature, oxygen saturation or blood pressure of a vehicle occupant can be determined from the image data with processes known to the person skilled in the art.

The autonomous vehicle 2 also comprises a user interface 26 (HMI=Human Machine Interface), which enables a vehicle occupant to interact with one or more vehicle systems. This user interface 26 can comprise an electronic display, for example (e.g. a GUI=graphical user interface) for outputting a graphic, symbols and/or content in text form, and an input interface for receiving an input (e.g. a manual input, speech input, and inputs through gestures, head or eye movements). The input interface can comprise keyboards, switches, touchscreens, eye trackers, etc. By way of example, a personal user profile can be set by a user via the user interface 26. The selection can take place, for example, via a dropdown menu, by means of which the user can select a user profile from a predefined list of user profiles by tapping a touchscreen, or by pressing keys on the user interface. The user can also transmit user authentication data to a server or a cloud, on which numerous user profiles are provided. Based on the user authentication data, the relevant configuration data for the vehicle can be selected from the user profile, and transmitted to the vehicle via a communication link.

The autonomous vehicle 2 also comprises a microphone 23, and means for speech recognition and speech analysis. Speech analysis while driving likewise allows conclusions to be drawn regarding the well being of the vehicle occupants.

The autonomous vehicle 2 also comprises a communication interface 19 for mobile networks. This comprises, e.g., a SIM card, with which the control unit can communicate via a mobile network, e.g. UMTS or LTE. The communication interface 19 can enable communication with a cloud, for example.

The autonomous vehicle 2 also comprises a communication interface 22 for an external user device, e.g. a wireless WLAN or Bluetooth interface, or a hard-wired USB connection. The communication interface 22 for an external user device is used for connecting to user devices such as smartphones, smartwatches, etc.

The communication interfaces 22 and/or 19 can also provide interfaces for the exchange of information and data between motor vehicles (C2C, car-to-car communication) or between vehicles and a traffic infrastructure (C2X, X2C).

As is described below in reference to exemplary embodiments, the control unit 18 for autonomous driving continuously receives physiological measurements regarding one or more vehicle occupants from a portable user device (1 in FIG. 1) via the communication interface 22. The control unit for autonomous driving 18 is configured to rate driving maneuvers on the basis of these physiological measurements, which is carried out at point in time that correlates with the point in time at which the physiological measurements by the portable user device 1 are recorded.

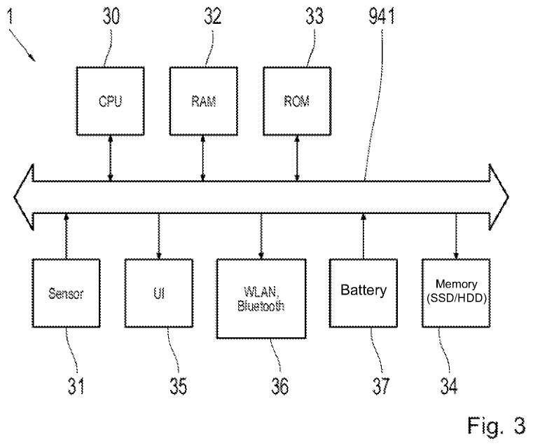

FIG. 3 shows a block diagram that illustrates an exemplary configuration of a portable user device 1, wherein the user device is configured to provide physiological measurements. The portable user device 1 comprises a processor 30. The processor 30 can be a computing unit, e.g. a central processing unit (CPU), which executes program instructions. The portable user device 1 also comprises memory components, e.g. a random access memory 32 (RAM) or a read-only memory 33 (ROM), and input/output interfaces that are functionally connected thereto. The RAM 32 stores programs used by the processor 30 for determining specific parameters. The information and current data necessary for executing the program that is stored therein is read out in the direct access ROM 33. It is likewise possible to connect an external drive 34, e.g. an external hard disk (hard disk drive: HDD), a flash memory, or a non-volatile solid state drive (SSD).

The portable user device 1 also comprises one or more biosensors 31, which are configured to record physiological measurements regarding a vehicle occupant. These preferably comprise one or more of the following sensors: a sensor for determining the heart rate and the blood oxygen saturation, wherein these sensors are preferably formed by an optical sensor (e.g. a photoplethysmography sensor), a sensor for measuring the electrical conductance of the skin, in particular the electro dermal activity, a sensor for measuring the temperature or a heat flow, a sensor for measuring respiration, a sensor for monitoring blood pressure, a sensor for monitoring muscle tone, etc.

The sensors can be in a single portable user device 1, which can be worn, for example, as a bracelet, a finger clip, or on the ear. Alternatively, numerous user devices can provide physiological measurements to the control unit for autonomous driving.

The portable user device 1 comprises a communication interface 36, e.g. a wireless transmitter, with which the recorded data can be transmitted wirelessly, in particular via UMTS, WLAN or Bluetooth, to the control unit for autonomous driving 18 (see FIG. 4) via the communication interface 22 and the vehicle communication network 28 of the autonomous vehicle 2.

The portable user device 1 also comprises a power source 37. The power source 37 is preferably in the form of a battery, and supplies the components connected thereto with electrical energy. The portable user device 1 also has a user interface 35 (UI), by means of which the user can read or input information. This user interface 35 can comprise a display, for example, in particular an LED monitor, which can indicate a measured physiological state to the wearer of the portable user device 1.

FIG. 4 shows a block diagram illustrating an exemplary configuration of a control unit for autonomous driving 18 (ECU 4). The control unit for autonomous driving 18 can be a control unit (electronic control unit, ECU, or electronic control module, ECM), for example. The control unit for autonomous driving 18 (ECU 4) comprises a processor 40. The processor 40 can be a computing unit, e.g. a central processing unit (CPU), which executes program instructions.

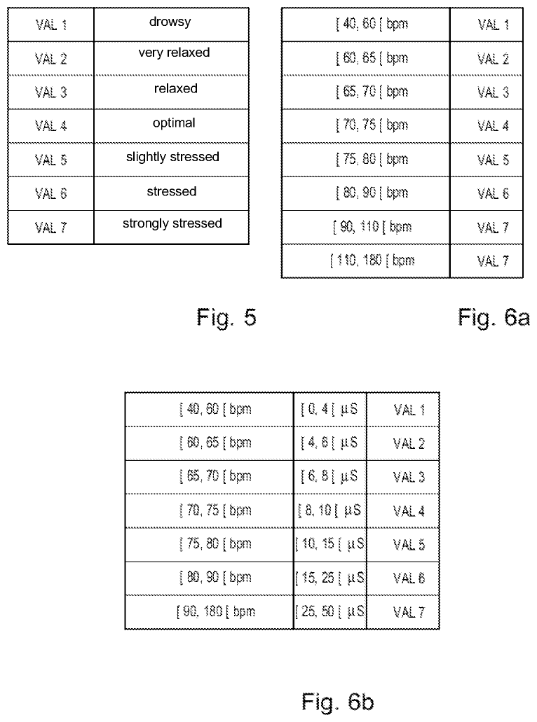

The processor of the control unit for autonomous driving 18 is configured to evaluate an executed or upcoming driving maneuver in that a rating is assigned thereto. The rating can be defined, e.g., by a parameter, a numerical value, etc. By way of example, various rating values can be predefined in the control unit, wherein each rating value is assigned to a well being of a vehicle occupant (see FIG. 5).

The control unit for autonomous driving 18 also comprises a memory and an input/output interface. The memory can be composed of one or more non-volatile computer-readable media, and comprises at least one program storage region and one data storage region. The program storage region and the data storage region can comprise combinations of various types of memory, e.g. a read-only memory 43 (ROM) and a random access memory 42 (RAM) (e.g. dynamic RAM (“DRAM”), synchronous DRAM (“SDRAM”) etc.). The control unit for autonomous driving 18 can also comprise an external memory 44, e.g. an external hard disk drive (HDD), a flash memory drive, or a non-volatile solid state drive (SDD).

The control unit for autonomous driving 18 also comprises a communication interface 45, via which the control unit can communicate with the vehicle communication network (28 in FIG. 2).

FIG. 5 shows predefined rating values VAL1-VAL7, which are assigned to specific states of being of the vehicle occupant according to an exemplary embodiment of the invention. The rating values VAL1-VAL7 are stored, for example, in a memory (44 in FIG. 4) of the control unit (18 in FIG. 2) for autonomous driving. A predefined rating value VAL1 is assigned to a state of being, “drowsiness.” A predefined rating value VAL2 is assigned to a state of being, “very relaxed.” A predefined rating value VAL3 is assigned to a state of being, “relaxed.” A predefined rating value VAL4 is assigned to a state of being, “optimal.” A predefined rating value VAL5 is assigned to a state of being, “slightly stressed.” A predefined rating value VAL6 is assigned to a state of being, “stressed.” A predefined rating value VAL7 is assigned to a state of being, “very stressed.” By defining these rating values such as stress, anger, drowsiness, boredom, etc., can be quantified and assigned to driving maneuvers, as explained in greater detail below. If the vehicle is to be operated economically and comfortably while underway, for example, but the occupants are in a hurry, such that they are irritated by the “peaceful” manner of driving, the vehicle can rate slow driving maneuvers with the rating values VAL1 to VAL3 as not optimal, based on physiological measurement, and thus modify the manner of driving to a more sporty profile.

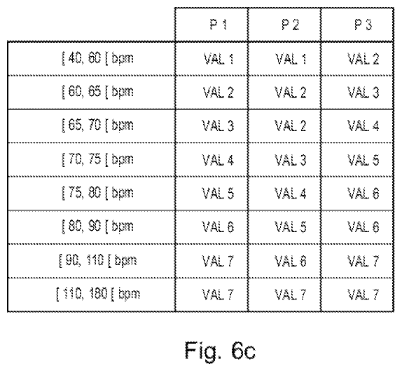

FIG. 6a shows how various ranges of a heart rate recorded by the portable user device 1 are assigned the aforementioned predefined rating values VAL1-VAL7 according to an exemplary embodiment of the invention. A heart rate between 40 and 60 beats per minute (bpm) is assigned the rating value VAL1; a heart rate between 60 and 65 bpm is assigned the rating value VAL2; a heart rate between 65 and 70 bpm is assigned the rating value VAL3; a heart rate between 70 and 75 bpm is assigned the rating value VAL4; a heart rate between 75 and 80 bpm is assigned the rating value VAL5; a heart rate between 80 and 90 bpm is assigned the rating value VAL6; a heart rate between 90 and 110 bpm is assigned the rating value VAL7, and a heart rate between 110 and 180 is likewise assigned the rating value VAL7. These assignments can also be stored in advance in the memory (44 in FIG. 4) of the control unit (18 in FIG. 2) for autonomous driving. The assignment can be user-specific. By way of example, the assignment can be based on the age of the vehicle occupant, and stored in a respective user profile (see exemplary embodiment in FIG. 6c).

FIG. 6a shows an exemplary assignment to a specific person. The assignment can be predefined by the control unit for autonomous driving, or it can be created dynamically (learned). The rating algorithm can potentially first determine the normal parameter (e.g. a resting pulse), in order to be able to make an assignment to states of being.

The well being of a vehicle occupant can also be expressed by an increased adrenaline level, a specific posture, a change in skin conductance, a change in oxygen saturation in blood, etc.

Numerous types of measurements can also be recorded, and a conclusion regarding the state of being of the vehicle occupant can be drawn from a combined view of all of the measurements. By way of example, predefined multidimensional tables can be stored for this, or rating values can be calculated analytically from numerous measurements through computing. Different priorities can be assigned in the assignment of the rating values if there are numerous measurement variables of the physiological measurements. By way of example, the measurement of the heart rate can be given a higher priority than the measurement of skin conductance.

FIG. 6b shows, by way of example, how various ranges of a heart rate recorded by the portable user device 1 are assigned in combination with a measured skin conductance to the aforementioned predefined rating values (VAL1-VAL2) according to an alternative exemplary embodiment of the invention. By way of example, a heart rate of 40 to 60 bpm in combination with a skin conductance between 0 and 4 microsiemens is assigned the rating value VAL1; a heart rate of 60 to 65 bpm in combination with a skin conductance between 4 and 6 microsiemens is assigned the rating value VAL2; a heart rate of 65 to 70 bpm in combination with a skin conductance between 6 and 8 microsiemens is assigned the rating value VAL3, etc.

FIG. 6c shows how various ranges of a heart rate are assigned rating values in a user-specific manner according to another alternative exemplary embodiment of the invention. A heart rate of 40 to 60 bpm is assigned the rating value VAL1 for a user P1 and a user P2, and the rating value VAL2 for a user P3; a heart rate of 60 to 65 bpm is assigned the rating value VAL2 for user P1 and user P2, and the rating value VAL3 for user P3; a heart rate of 65 to 70 bpm is assigned the rating value VAL3 for user P1, rating value VAL2 for user P2, and rating value VAL4 for user P3; a heart rate of 70 to 75 bpm is assigned the rating value VAL4 for user P1, rating value VAL3 for user P2, and rating value VAL5 for user P3; a heart rate of 75 to 80 bpm is assigned the rating value VAL5 for user P1, rating value VAL4 for user P2, and rating value VAL6 for user P3; a heart rate of 80 to 90 bpm is assigned the rating value VAL6 for user P1, rating value VAL5 for user P2, and rating value VAL6 for user P3; a heart rate of 90 to 110 bpm is assigned rating value VAL7 for user P1, rating value VAL6 for user P2, and rating value VAL7 for user P3; and a heart rate of 110 to 180 bpm is assigned the rating value VAL7 for users P1, P2 and P3. These assignments can also be stored in a memory (44 in FIG. 4) of the control unit (18 in FIG. 2) for autonomous driving. By way of example, the assignment can be based on the ages of the vehicle occupants, and stored in a respective user profile. Alternatively, these assignments can also be based on numerous parameters, e.g. age, sex, body fat percentage, existing illnesses, etc. The same applies for gestures, skin color and other physiological variables.

FIG. 6c shows how various ranges of a heart rate are assigned driving mode-specific rating values according to yet another alternative exemplary embodiment. Two different driving modes are assumed, a first mode SET1, which is an economical driving mode, and a second mode SET2, which is a sporty driving mode. The driving mode can be set, for example, by a vehicle occupant by means of a user interface. A heart rate between 40 and 60 bpm is assigned the rating value VAL1 for a driving mode SET1 and also assigned the rating value VAL1 for a driving mode SET2; a heart rate between 60 and 65 bpm is assigned the rating value VAL 2 for the driving mode SET1 and likewise assigned the rating value VAL2 for the driving mode SET2; a heart rate between 65 and 70 bpm is assigned the rating value VAL3 for the driving mode SET1 and the rating value VAL2 for the driving mode SET2; a heart rate between 70 and 75 bpm is assigned the rating value VAL4 for the driving mode SET1 and the rating value VAL3 for the driving mode SET2; a heart rate between 75 and 80 bpm is assigned the rating value VAL5 for the driving mode SET1 and the rating value VAL4 for the driving mode SET2; a heart rate between 80 and 90 bpm is assigned the rating value VAL6 for the driving mode SET1 and the rating value VAL5 for the driving mode SET2; a heart rate between 90 and 110 bpm is assigned the rating value VAL7 for the driving mode SET1 and the rating value VAL6 for the driving mode SET2; a heart rate between 110 and 180 bpm is assigned the rating value VAL7 for the driving mode SET1 and likewise the rating value VAL7 for the driving mode SET2. These assignments can also be stored in advance in a memory (44 in FIG. 4) of the control unit (18 in FIG. 2) for autonomous driving. It can be derived from the table that in the sporty driving mode, the optimal rating is at a higher heart rate (in this case 75-80 bpm) than in the economical driving mode (in this case 70-75 bpm).

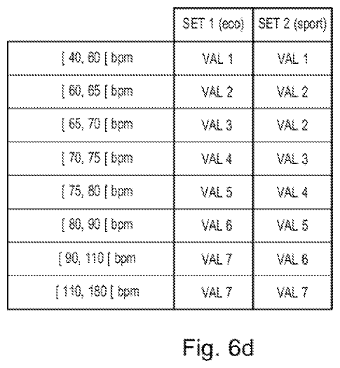

FIG. 7a shows a schematic illustration of numerous defined driving maneuver variations M1a to M1e of a driving maneuver M1 according to an exemplary embodiment of the invention. In the presented exemplary embodiment, the driving maneuver M1 corresponds to cornering, and the driving maneuver variations M1a to M1e correspond to cornering with a predefined transverse acceleration. A driving maneuver M1a corresponds to cornering with a transverse acceleration of 2 m/s2; a driving maneuver M1b corresponds to cornering with a transverse acceleration of 3 m/s2; a driving maneuver M1c corresponds to cornering with a transverse acceleration of 4 m/s2; a driving maneuver M1d corresponds to cornering with a transverse acceleration of 5 m/s2; and a driving maneuver M1e corresponds to cornering with a transverse acceleration of 6 m/s2.

In the above example, the driving maneuvers M1a-M1e are determined solely by the transverse acceleration and the context, “cornering.” In alternative exemplary embodiments, numerous vehicle operating parameters that are recorded by the vehicle sensors can also be drawn on to define a driving maneuver.

FIG. 7b shows a schematic illustration of numerous predefined driving maneuver variations M2a to M2g of a driving maneuver M2. In the present case, the driving maneuver M2 corresponds to highway driving on the, and the driving maneuver variations M2a to M2g correspond to highway driving at a predefined speed. A driving maneuver M2a corresponds to highway driving at a speed of 100 km/h; a driving maneuver M2b corresponds to highway driving at a speed of 110 km/h; a driving maneuver M2c corresponds to highway driving at a speed of 120 km/h; a driving maneuver M2d corresponds to highway driving at a speed of 130 km/h; a driving maneuver M2e corresponds to highway driving at a speed of 140 km/h; a driving maneuver M2f corresponds to highway driving at a speed of 150 km/h; and a driving maneuver M2g corresponds to highway driving at a speed of 160 km/h.

In the above example, the driving maneuvers M2a-M2g are determined solely by the speed and the context, “highway driving.” In alternative exemplary embodiments, numerous vehicle operating parameters can also be drawn on to define a driving maneuver. Moreover, the traffic conditions can also be taken in to account. Thus, parameters for the states of other vehicles relating to the environment can be drawn on, for example, in particular their positions, speeds or direction of travel, dangerous situations, etc. which are obtained via the vehicle-based communication system (e.g. a car-to-X interface and/or an X-to-car interface). Furthermore, the traffic congestion can be estimated using numerous sensors, e.g. radar, lidar and optical cameras, which may already be available on a vehicle as parts of other systems.

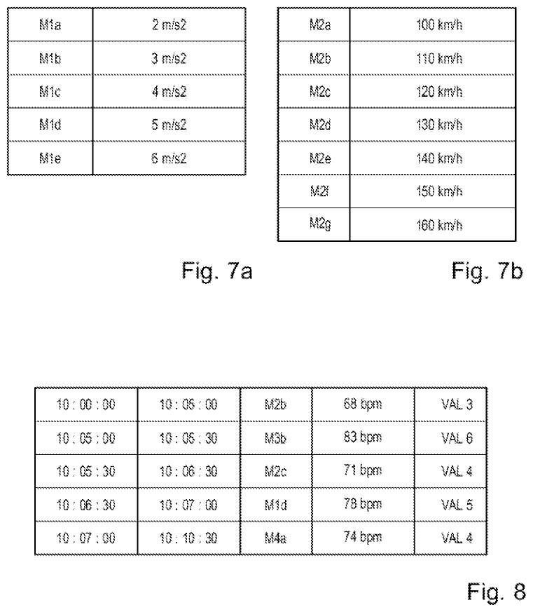

FIG. 8 shows an exemplary table comprising a temporal sequence of predefined driving maneuvers carried out by a vehicle in the framework of driving over a distance. The table represents the results of determining physiological measurements during a specific driving distance, lasting here from 10:00:00 to 10:10:30. The table comprises measurements for the heart rate, measured by the portable user device 1 at various points in time, correlating to the respective driving maneuvers. The driving maneuver is known to the control device for autonomous driving from the control and navigation context, and is determined in a manner known to the person skilled in the art. As can be seen in the table, a driving maneuver M2b (highway driving at a speed of 110 km/h, see FIG. 7b) takes place from time t=10:00:00 to time t=10:05:00, and the measured heart rate is 68 bpm. According to the predefined assignment (FIG. 6a), this heart rate is assigned the rating value VAL3, representing “relaxed.” A driving maneuver M3b (passing maneuver) takes place from time t=10:05:00 to time t=10:05:30, and the measured heart rate is 83 bpm. According to the predefined assignment (FIG. 6a), this heart rate is assigned the rating value VALE, representing “stressed.” A driving maneuver M2c (highway driving at a speed of 120 km/h, see FIG. 7b) takes place from the time t=10:05:30 to time t=10:06:30, and the measured heart rate is 71 bpm. According to the predefined assignment (FIG. 6a), this heart rate is assigned the rating value VAL4, representing “optimal.” A driving maneuver M1d (cornering with a transverse acceleration of 5 m/s2) takes place from the time t=10:06:30 to time t=10:07:00, and the measured heart rate is 78 bpm. According to the predefined assignment (FIG. 6a), this heart rate is assigned the rating value VAL5, representing “slightly stressed.” In the present exemplary embodiment, the driving maneuver M1d corresponds to cornering with a transverse acceleration of 5 m/s2, which takes place when exiting the highway. The control unit of the autonomous vehicle has the task of exiting the highway at the next exit. The vehicle changes lanes, to the deceleration lane on the right. Shortly before the curve on the exit starts, the vehicle is strongly decelerated. The transverse acceleration in the curve is relatively high due to the late deceleration, such that a vehicle occupant feels uneasy, and his heart rate increases to 78 bpm. A driving maneuver M4a (road driving at a speed of 70 km/h) takes place from time t=10:07:00 to time t=10:10:30, and the measured heart rate is 74 bpm. According to a predefined assignment (FIG. 6a), this heart rate is assigned the rating value VAL4, representing “optimal.”

The measurement for the heart rate can also be a mean value of numerous measurements that have been taken at the same time as a specific driving maneuver.

The associated rating values VAL1 to VAL7 are thus determined by the control unit for autonomous driving from the obtained physiological measurements based on predefined ratings, which represent the emotional states such as relaxed or stressed, or drowsiness, and thus evaluating the driving maneuver that has been executed. As such, the control unit for autonomous driving 18 can determine, based on the recorded physiological measurements, whether the driver is relaxed or tense, or even stressed.

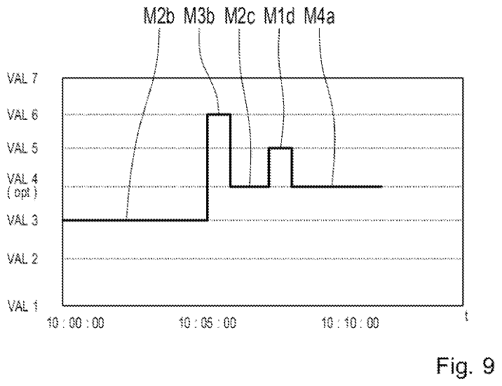

FIG. 9 shows a graph of the rating values determined by the control unit for autonomous driving (18 in FIG. 2) for the temporal sequence of driving maneuvers in FIG. 8. From the time t=10:00:00 to time t=10:05:00, the driving maneuver M2b (highway driving at a speed of 110 km/h, see FIG. 7b) is registered, which is assigned the rating value VAL3, representing “relaxed.” From time t=10:05:00 to time t=10:05:30, a driving maneuver M3b (passing maneuver) is registered, which is assigned the rating value VALE, representing “stressed.” From the time t=10:05:30 to time t=10:06:30, a driving maneuver M2c (highway driving at a speed of 120 km/h, see FIG. 7b) is registered, which is assigned the rating value VAL4, representing “optimal.” From time t=10:06:30 to time t=10:07:00, a driving maneuver M1d (cornering with a transverse acceleration of 5 m/s2, see FIG. 7a) is registered, which is assigned the rating value VAL5, representing “slightly stressed.” From time t=10:07:00 to time t=10:10:30, a driving maneuver M4a (road driving at a speed of 70 km/h) is registered, which is assigned the rating value VAL4, representing “optimal.”

In this manner, a rating of the driving maneuvers of the control unit for autonomous driving can take place on the basis of the measured physiological measurements of one or more vehicle occupants, e.g. continuously or during special training or learning phases. The rating of various driving maneuvers that are carried out obtained in this manner can be used to create one or more user profiles.

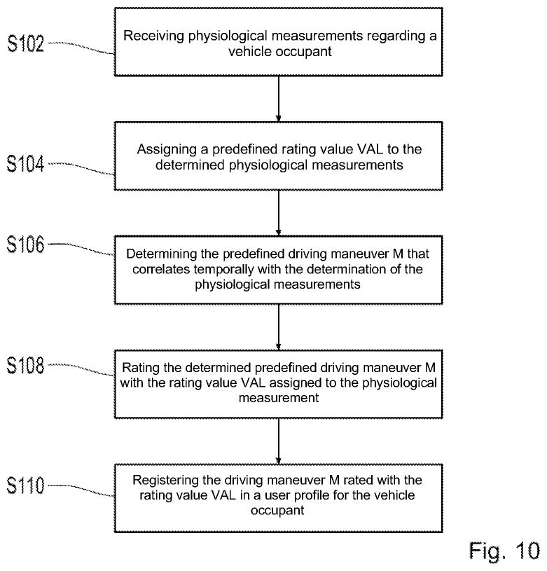

FIG. 10 shows a flow chart illustrating the creation of a user profile by the control unit for autonomous driving. In step S102, the control unit for autonomous driving receives physiological measurements regarding a vehicle occupant. In step S104, the control unit for autonomous driving assigns the recorded physiological measurements to a respective predefined rating value VAL. In step S106, the control unit for autonomous driving determines a driving maneuver M, which correlates temporally to the determination of the physiological measurements. In step S108, the control unit for autonomous driving rates the detected driving maneuver M with the rating value VAL assigned to the physiological measurement. In step S110, the control unit for autonomous driving registers the rated driving maneuver M in a user profile of the vehicle occupant.

In the above exemplary embodiment shown in FIG. 10, the physiological measurements are rated in an absolute manner. In alternative exemplary embodiments, the physiological measurements can also be rated in a relative manner (based on changes). After a driving maneuver has been completed, the physiological measurements (biological data, gestures and/or speech by vehicle occupants) can be analyzed as described above.

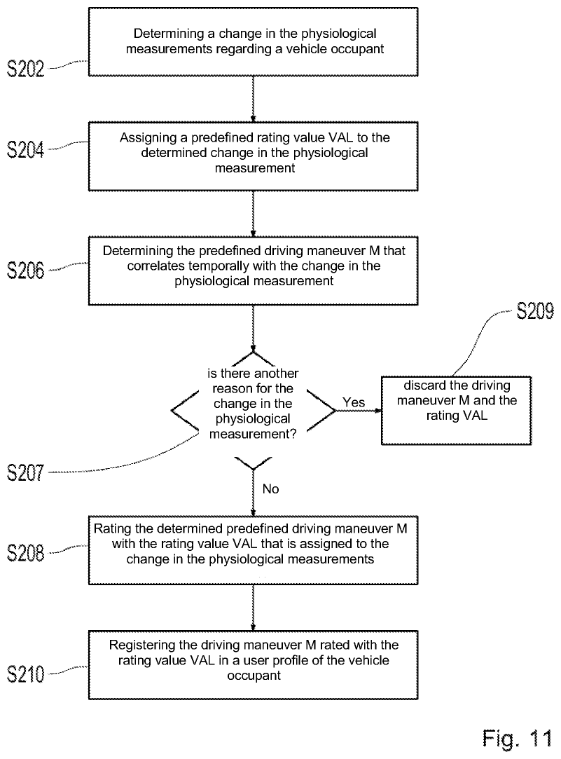

FIG. 11 shows a flow chart illustrating a method for creating a user profile by the control unit for autonomous driving, in which the physiological measurements are rated in a relative manner (based on changes). After a maneuver, the heart rate, for example, can increase when the vehicle occupant is frightened, the blood pressure can change, the posture reflects the well being (cramping, holding fast, relaxed “sagging,” nervous behavior, etc.), the skin color changes accordingly, etc. The control unit for autonomous driving can use this information regarding changes in the well being of one or more vehicle occupants, and adjust the manner of driving accordingly (e.g. more/less distance to vehicles in front, earlier/later braking while cornering, lower/higher cornering speeds, sporty manner of driving).

In step S202, the control unit for autonomous driving determines a change in the physiological measurements regarding a vehicle occupant. In step S204, the control unit for autonomous driving assigns the determined change in the physiological measurement to a predefined rating value VAL. When the physiological measurements do not change, or change only slightly, or there are no noticeable gestures or speech, the maneuver can be rated well. If the physiological measurements change to a greater extent, it is determined whether the driving maneuver is the reason for the change. In a step S206, the control unit for autonomous driving detects a driving maneuver M that correlates temporally to the determined change in the physiological measurements. In step S207, the control unit for autonomous driving checks whether there is another reason for the change in the physiological measurements. If it is determined in step S207 that there is no other reason, the control unit for autonomous driving rates the detected driving maneuver M in step S208 with the rating value VAL that is assigned to the determined change in the physiological measurements. In step S210, the control unit for autonomous driving registers the rated driving maneuver M in a user profile of the vehicle occupant. If instead it is determined in step S207 that there is another reason, the driving maneuver M and the rating VAL are discarded in step S209.

The reason can likewise be explained with gestures and speech. If the driving maneuver is the only possible reason, a corresponding rating can be defined on the basis of the change or expression. If instead it is determined with means such as image or speech analysis that the occupant is in an emotionally tense conversation, the control unit for autonomous driving can decide that the associated physiological measurements are not to be drawn on for rating driving maneuvers.

Through a continuous analysis of all of the maneuvers for all of the vehicle occupants, user profiles can be created with ratings for all driving maneuvers. As a result, it is possible for the control unit for autonomous driving to anticipate how specific types of people will react during specific maneuvers, i.e. how the maneuvers are “received” by the vehicle occupants. Occupants rate manners of driving differently. The aim can be to adapt the manner of driving such that different people always feel safe.

Based on the user profile created in this manner, the control unit for autonomous driving can then rate an upcoming driving maneuver or driving maneuver variation. The selection of the appropriate manner of driving (speeds, transverse accelerations, maneuvers: evasive maneuvers, braking, or combinations of accelerations and decelerations, position in the driving lane: left, right, middle, . . . ) is carried out in this manner by the autonomous vehicle itself.

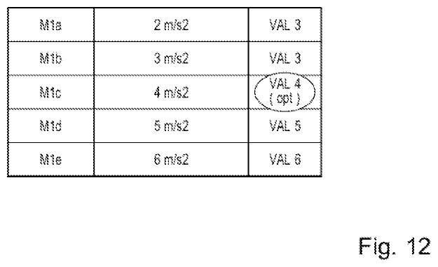

FIG. 12 shows a table with a user profile for the driving maneuver variations M1a-M1e of the driving maneuver category M1, “cornering,” according to the present exemplary embodiment, created by the control unit 18 for autonomous driving. As can be seen in the table, a driving maneuver variation M1a, “cornering with a transverse acceleration of 2 m/s2” is assigned the predefined rating value VAL3 as a rating value; a driving maneuver variation M1b, “cornering with a transverse acceleration of 3 m/s2” is assigned the predefined rating value VAL3 as a rating value; a driving maneuver variation M1c, “cornering with a transverse acceleration of 4 m/s2” is assigned the predefined optimal rating value VAL4 as a rating value; a driving maneuver variation M1d, “cornering with a transverse acceleration of 5 m/s2” is assigned the predefined rating value VAL5 as a rating value (cf. FIG. 8 and FIG. 9); and a driving maneuver variation M1e, “cornering with a transverse acceleration of 6 m/s2” is assigned the predefined rating value VALE as a rating value.

The user profile that has been created is then used by the control unit for autonomous driving 18 to control upcoming driving maneuvers such that the driving behavior of the vehicle is optimized. The manner of driving is thus adapted to the people in the vehicle. Accordingly, when an upcoming driving maneuver of the driving maneuver category M1, “cornering,” is to be carried out, the control unit for autonomous driving selects the driving maneuver variation M1c, “cornering with a transverse acceleration of 4 m/s2,” which is assigned the optimal rating value VAL4 as a rating value.

The control unit 18 for autonomous driving according to the present invention can store the created user profile, and reuse it later in order to optimize the driving behavior of the vehicle.

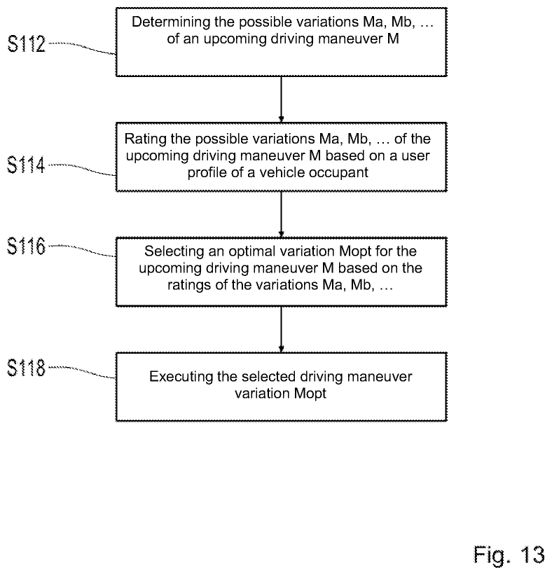

FIG. 13 shows a flow chart illustrating the rating of possible driving maneuver variations of an upcoming driving maneuver by the control unit for autonomous driving 18 based on a user profile of a vehicle occupant. In step 112, the control unit for autonomous driving determines possible variations Ma, Mb, Mc, etc. of a driving maneuver M that is to be carried out. In step 114, these driving maneuver variations are rated on the basis of a user profile of a vehicle occupant that was created earlier. In step 116, an optimal variation Mopt is selected for the upcoming driving maneuver M, based on the ratings of the driving maneuver variations. In step 118, the control unit for autonomous driving controls a control unit for a steering system, a control unit for a braking system, and/or a control unit for a drive train in accordance with the selected optimal driving maneuver variation, such that the selected driving maneuver is carried out.

In the above exemplary embodiment, the control unit for autonomous driving selects optimal driving maneuver variations in each case from the perspective of the well being of the occupants. In alternative exemplary embodiments, the control unit for autonomous driving can also decide between the well being of the vehicle occupants and the demands of a traffic situation, based on the traffic situation. The control unit for autonomous driving can thus decide during a passing maneuver that it is appropriate in the current traffic situation to accept a certain amount of stress for the vehicle occupant(s), in order to quickly complete the passing maneuver. In this manner, a parameter can be determined for every type of driving maneuver, which determines the extent to which the well being of the occupants should affect the selection of the driving maneuver variation.

On the basis of the rating of the driving maneuver, or on the basis of information regarding the health of a vehicle occupant, certain maneuvers for specific occupants can also be replaced by other maneuvers. By way of example, the control unit for autonomous driving can detect an obstruction on the street and identify “avoiding collision with the obstacle” as an upcoming driving maneuver variation. For this driving maneuver, the control unit for autonomous driving can identify two possible driving maneuver variations, specifically “driving around the obstacle” as the first driving maneuver variation, and “braking before reaching the obstacle” as the second driving maneuver variation. Depending on the information in the user profile, certain maneuvers can then be avoided, and replaced by other possible maneuvers. By way of example, it can be derived from the user profile that a vehicle occupant normally reacts negatively to the driving maneuver variation, “driving around the obstacle,” but positively to the driving maneuver variation, “braking before reaching the obstacle.” Consequently, the control unit for autonomous driving can select the preferred driving maneuver variation, “braking before reaching the obstacle” here. In this manner, the control unit for autonomous driving replaces the driving maneuver variation “driving around the obstacle” with the driving maneuver variation, “braking before reaching the obstacle,” for the vehicle occupants in question.

Information regarding the health of a vehicle occupant can also be stored in a user profile, and certain driving maneuvers can be avoided, depending on the state of health. The state of health can affect the rating of a driving maneuver. The manner of driving can be rated based on the heath data. The manner of driving can be continuously optimized with this data. If the control device for autonomous driving detects an ill vehicle occupant in the vehicle, for example, then those driving maneuvers that present a difficulty for the vehicle occupant, such as cornering with high transverse accelerations, etc. are systematically given a worse rating by the control unit for autonomous driving. In this manner, health data can have an effect on the driving manner of the vehicle. The system can thus be pre-conditioned (setting a specific driving manner when people get in the vehicle). III, old, sensitive people can be brought to their destination very gently (with low transverse and longitudinal accelerations). Vehicle occupants that are stressed can be reassured by a specific driving manner that has been set and adapted to their needs. Music and lighting in the vehicle can likewise be adapted on the basis of the health data. The aim is to calm the occupants, and bring them safely and happily to their destination.

According to another aspect of the invention, injured people or pregnant women in pain can be brought to a hospital as quickly as possible, using the shortest route, if the control unit for autonomous driving detects a corresponding state of health through analysis of the physiological measurements.

The ratings of driving maneuvers determined by the above exemplary embodiments, or the user profiles created on the basis thereof, can also be used to further optimize the driving manner, e.g. through the use of deep learning, artificial intelligence (AI) or machine learning. The driving manner can be adapted incrementally, e.g. in steps. The driving manner can thus be subdivided into steps, and adapted in a noticeably quick manner. The driving manner can also be continuously adapted, i.e. modified in small steps. The driving manner can also be adapted in a combination of incremental and continuous modifications.

The control unit for autonomous driving can also be configured such that the continuous optimization of the driving manner can be switched on or off by means of a user interface (HMI=human-machine interface) (user interface 26 in FIG. 2). The vehicle occupants can decide whether the driving manner is optimized incrementally, continuously, in both manners, or not at all, through speech, gestural, or haptic inputs, etc. The range of optimization can be limited by the occupants. They can select, a “comfortable/economical driving manner,” for example, via the user interface. This can take place, for example, when inputting the destination for the autonomous vehicle, by means of speech, gestural, or haptic input, etc. The driving manner can thus be selected at the start. The control unit for autonomous driving then optimizes the driving manner accordingly, in that it prioritizes “more comfortable” or “more economical” driving maneuvers through better ratings. Alternatively, the vehicle occupant can select a “sporty” driving manner. The control unit for autonomous driving would then give more sporty driving maneuvers a better rating, thus prioritizing them. The focus of the optimization is defined by the vehicle occupants via the user interface. Undesired optimization of the driving manner by the control unit for autonomous driving can be avoided with these optional possibilities for setting the driving manner.

The acquired data (user profile) can also be exported from the control unit for autonomous driving and used, e.g., in other vehicles. The user profile can thus also be stored in a central memory, e.g. on a server or in a cloud server. A user profile in the control unit for autonomous driving can also be synchronized with a central user profile. Data from the user profile can be sent to the manufacturer, e.g. for longterm studies and further analyses. Collected data can be used as training data for AI algorithms. The vehicle control device or the user end device—insofar as aspects thereof are outsourced to a server or the cloud—can send the data to a central unit (e.g. the manufacturer), and the manufacturer can thus draw conclusions regarding the algorithm as to how it is received in a large sampling and whether it has reached the limits of its capabilities (the vehicle should behave differently, but is unable to adapt because it has already reached the limits of the function). This has the advantage that the manufacturer can then further develop the algorithm, and offer an update for the control logic in the vehicle.

### REFERENCE SYMBOLS

1 portable user device
2 autonomous vehicle
12 steering system control device
14 braking system control device
16 drive train control device
18 control device for autonomous driving
19 communication interface
20 environment sensors
21 interior camera
22 communication interface
23 microphone
24 satellite navigation unit
26 user interface
28 vehicle communication network
30 portable user device processor
31 biosensor
32 RAM memory
33 ROM memory
34 memory drive
35 user interface
36 communication interface
37 power source
40 control unit for autonomous driving processor
42 RAM memory
43 ROM memory
44 memory drive
46 user interface

1. A control device for autonomous driving comprising a processor configured to rate an executed or upcoming driving maneuver (M1-M4) based on physiological measurements.

2. The control device for autonomous driving according to claim 1, wherein the physiological measurements comprise a heart rate, skin conductance, oxygen saturation in blood, blood pressure, and/or adrenaline concentration of a vehicle occupant.

3. The control device for autonomous driving according to claim 1, wherein the physiological measurements comprise information obtained through image analysis or speech analysis.

4. The control device for autonomous driving according to claim 1, wherein the processor is configured to create a user profile based on rated driving maneuvers.

5. The control device for autonomous driving according to claim 4, wherein the processor is configured to rate an upcoming driving maneuver based on the created user profile.

6. The control device for autonomous driving according to claim 1, wherein the processor is configured to rate numerous variations of a driving maneuver, and select an optimal variation based on the rating.

7. The control device for autonomous driving according to claim 1, wherein the physiological measurements are given different priori-ties.

8. The control device for autonomous driving according to claim 1, wherein the processor is configured to identify relative changes in the physiological measurements, and rate a driving maneuver based on the relative changes in the physiological measurements.

9. The control device for autonomous driving according to claim 1, wherein the processor is configured to check whether a change in the physiological measurements can be attributed to a driving maneuver or some other reason, and to then rate the driving maneuver when the driving maneuver can be assumed to be the reason for the change.

10. A method for autonomous driving, in which an executed or upcoming driving maneuver is rated on the basis of physiological measurements.

11. The control device for autonomous driving according to claim 2, wherein the processor is configured to create a user profile based on rated driving maneuvers.

12. The control device for autonomous driving according to claim 2, wherein the processor is configured to rate numerous variations of a driving maneuver, and select an optimal variation based on the rating.

13. The control device for autonomous driving according to claim 2, wherein the physiological measurements are given different priori-ties.

14. The control device for autonomous driving according to claim 2, wherein the processor is configured to identify relative changes in the physiological measurements, and rate a driving maneuver based on the relative changes in the physiological measurements.

15. The control device for autonomous driving according to claim 2, wherein the processor is configured to check whether a change in the physiological measurements can be attributed to a driving maneuver or some other reason, and to then rate the driving maneuver when the driving maneuver can be assumed to be the reason for the change.

16. The control device for autonomous driving according to claim 3, wherein the processor is configured to create a user profile based on rated driving maneuvers.

17. The control device for autonomous driving according to claim 3, wherein the processor is configured to rate numerous variations of a driving maneuver, and select an optimal variation based on the rating.

18. The control device for autonomous driving according to claim 3, wherein the physiological measurements are given different priori-ties.

19. The control device for autonomous driving according to claim 3, wherein the processor is configured to identify relative changes in the physiological measurements, and rate a driving maneuver based on the relative changes in the physiological measurements.

20. The control device for autonomous driving according to claim 3, wherein the processor is configured to check whether a change in the physiological measurements can be attributed to a driving maneuver or some other reason, and to then rate the driving maneuver when the driving maneuver can be assumed to be the reason for the change.
